(19) United States
(12) Patent Application Publication
(10) Pub. No.: US 2025/0041246 A1
(43) Pub. Date: Feb. 6, 2025

(54) METHODS AND COMPOSITIONS FOR TREATING VITILIGO

(71) Applicants: THE REGENTS OF THE UNIVERSITY OF CALIFORNIA, Oakland, CA (US); TRUSTEES OF TUFTS COLLEGE, Boston, MA (US)

(72) Inventors: Anand Ganesan, Irvine, CA (US); Jessica Shiu, Irvine, CA (US); Mihaela Balu, Irvine, CA (US); Qing Nie, Irvine, CA (US); Irene Georgakoudi, Medford, MA (US)

(21) Appl. No.: 18/714,530

(22) PCT Filed: Nov. 30, 2022
(86) PCT No.: PCT/US2022/080696

§ 371 Date: May 29, 2024

Related U.S. Application Data
(60) Provisional application No. 63/284,171, filed on Nov. 30, 2021.

(51) Int. Cl. A61K 31/155; A61K 45/06

(52) U.S. Cl. CPC A61K 31/155; A61K 45/06

(57) ABSTRACT

Methods for treating vitiligo in a patient in need thereof, methods of inducing repigmentation in skin, methods for preventing further loss of pigmentation in a patient having vitiligo, and methods of reducing symptoms of vitiligo, wherein the methods feature topically administering a composition to a target area of skin tissue of the patient affected by vitiligo, wherein the composition induces glycolysis in at least a portion of the target area of skin tissue administered the composition. The composition may help treat vitiligo, symptoms thereof, prevent further loss of pigmentation, and/or induce repigmentation in skin.

### CROSS-REFERENCES TO RELATED APPLICATIONS

This application claims benefit of U.S. Provisional Application No. 63/284,171 filed Nov. 30, 2021, the specification of which is incorporated herein in their entirety by reference.

### STATEMENT REGARDING FEDERALLY SPONSORED RESEARCH OR DEVELOPMENT

This invention was made with Government support under Grant Nos. 5P30AR075047-03, R01EB026705, and R21AR073408, awarded by the National Institutes of Health (NIH). The Government has certain rights in the invention.

### FIELD OF THE INVENTION

The present invention relates to melanocytic disorders, more specifically to methods and compositions for treating melanocytic disorders such as vitiligo.

### BACKGROUND OF THE INVENTION

Vitiligo is an autoimmune disorder that affects 4% of the population where the immune system attacks and destroys melanocytes, cells that produce pigment in the skin. It creates white patches that can be disfiguring, particularly for people of color, and can cause itching and burning of the skin. While active vitiligo results from the activation of T cells and the immune system, it is unclear why some vitiligo patches do not repigment even when there are no active immune cells in the skin. While topical JAK inhibitors are in development, they are only effective in facial lesions. Better treatments are needed for patients with stable vitiligo patches that are not responding to treatment.

### BRIEF SUMMARY OF THE INVENTION

It is an objective of the present invention to provide compositions and methods that allow for the treatment of stable vitiligo, as specified in the independent claims. Embodiments of the invention are given in the dependent claims. Embodiments of the present invention can be freely combined with each other if they are not mutually exclusive.

As used herein, the term melanocyte repopulation or repigmentation may refer to the stimulation of melanocytes to move to diseased skin (e.g., stimulating melanocytes to migrate to a particular region of the skin tissue). As used herein, the term increasing pigmentation refers to increasing pigment in cells already in the tissue. As used herein, “lesional tissue” refers to the depigmented tissue in a vitiligo patient, and “nonlesional tissue” refers to the normal appearing tissue in a vitiligo patient.

The present invention features methods for treating vitiligo in a patient in need thereof. In certain embodiments, the method comprises topically administering a composition to a target area of skin tissue of the patient affected by vitiligo, wherein the composition induces glycolysis and stimulates repigmentation in at least a portion of the target area of skin tissue that is administered the composition.

The present invention also features a method of inducing repigmentation in skin lacking melanocytes of a patient. In some embodiments, the method comprises topically administering a composition to a target area of skin tissue of the patient, wherein the composition induces glycolysis and stimulates repigmentation in at least a portion of the target area of skin administered the composition.

The present invention also features a method of preventing further loss of pigmentation in a patient having vitiligo. In some embodiments, the method comprises topically administering a composition to a target area of skin tissue of the patient, wherein the composition induces glycolysis and prevents further loss of pigmentation in at least a portion of the target area of skin tissue administered the composition.

The present invention also features a method of reducing symptoms of vitiligo. In some embodiments, the method comprises topically administering a composition to a target area of skin tissue of the patient affected by symptoms of vitiligo, wherein the composition induces glycolysis and reduces symptoms in at least a portion of the target area of skin tissue administered the composition.

Referring to the methods herein, in certain embodiments, the composition comprises a biguanide. In certain embodiments, the biguanide is phenformin. In some embodiments, the composition further comprises a pharmaceutically acceptable carrier. Pharmaceutically acceptable carriers are well known to one of ordinary skill in the art.

In certain embodiments, the method further comprises co-administering a secondary therapeutic. A non-limiting example of a secondary therapeutic is a JAK inhibitor. The secondary therapeutic may be administered at the same time as the composition. In certain embodiments, the secondary therapeutic is administered at a time before the composition is administered. In certain embodiments, the secondary therapeutic is administered at a time after the composition is administered.

The present invention also features a composition for use in a method of treating vitiligo, a method of inducing repigmentation in skin, a method of preventing further loss of pigmentation in a patient having vitiligo, or a method of reducing symptoms of vitiligo. The composition may comprise a glycolysis-inducing agent that stimulates glycolysis in skin cells having been contacted with the composition. In some embodiments, the glycolysis-inducing agent is a biguanide. In certain embodiments, the biguanide is metformin. In certain embodiments, the biguanide is phenformin. In certain embodiments, the composition further comprises a pharmaceutically acceptable carrier. In certain embodiments, the composition further comprises a secondary therapy. In certain embodiments, the secondary therapy is a JAK inhibitor.

The present invention also features a method of preparing a population of skin cells. In some embodiments, the method comprises harvesting melanocytes and keratinocytes from a portion of skin in a patient; and subjecting these skin cells to a treatment, wherein the treatment comprises a composition comprising a glycolysis-inducing agent, e.g., as described herein, e.g., a composition comprising a biguanide.

The present invention also features a method of transplanting skin cells. In certain embodiments, the method comprises harvesting melanocytes and keratinocytes from a portion of skin in a patient; and subjecting the skin cells to a treatment, wherein the treatment comprising a composition comprising a glycolysis-inducing agent, e.g., as described herein; and transplanting skin cells onto the patient.

One of the unique and inventive technical features of the present invention is the use of biguanides (e.g., metformin, phenformin) for treatment of stable vitiligo. Without wishing to limit the invention to any theory or mechanism, it is believed that the technical feature of the present invention advantageously provides for a reversal of the metabolic alterations in chronic vitiligo skin (e.g., reducing oxidative phosphorylation and inducing glycolysis). None of the presently known prior references or work has the unique inventive technical feature of the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

The features and advantages of the present invention will become apparent from a consideration of the following detailed description presented in connection with the accompanying drawings in which:

FIG. 1A, FIG. 1B, FIG. 1C and FIG. 1D shows in vivo multiphoton microscopy (MPM) images of vitiligo lesional and nonlesional skin and metabolic changes with depth independent of exposure. FIG. 1A shows representative en-face MPM images from the stratum granulosum in nonlesional (A1) and lesional skin (A2) and from the basal layer in nonlesional (B1) and lesional skin (B2) of one vitiligo patient. Average mitochondrial clustering (B) values based on z-stacks from all vitiligo patients as a function of depth for nonlesional (top right) and lesional (bottom right) skin are shown as spline fits. Error bars represent the standard deviation of the measurements for the images in all the z-stacks at each area. The labels A1, A2, B1, and B2 within the mitochondrial clustering panels represent the mitochondrial clustering values extracted from the panel's respective labeled images. Scale bars are 20 μm. FIG. 1B shows representative en-face MPM images from the stratum granulosum in sun-exposed (A1) and non sun-exposed skin (A2) and from the basal layer in sun-exposed (B1) and non sun-exposed skin (B2) of healthy volunteers. FIG. 1C shows the distribution of the median β values (left) and β variability values (right) in nonlesional and lesional skin of vitiligo patients; each value corresponds to a z-stack of images acquired in nonlesional and lesional skin. *=t-test p-value <0.05. FIG. 1D shows the distribution of the median β values (left) and B variability values (right) in sun-exposed and non sun-exposed skin of healthy volunteers; each value corresponds to a z-stack of images acquired in non sun-exposed and sun-exposed areas.

FIG. 2A, FIG. 2B, FIG. 2C, FIG. 2D, and FIG. 2E shows single cell RNA-seq analyses of lesional and nonlesional skin reveal unique keratinocyte cell states in vitiligo patients. FIG. 2A shows a schematic diagram of single cell isolation and scRNA-seq data analyses. FIG. 2B shows a UMAP (Uniform Manifold Approximation and Projection) plot of the cells from all patients in both nonlesional (left) and lesional skin (right). FIG. 2C shows feature plots showing the expression of the selected markers in the UMAP space of all cells. FIG. 2D shows a high density plot showing the relative gene expression of key marker genes in different cell subpopulations. Each density plot is composed of bar charts, and bar height corresponds to the relative expression level of the gene in cells that is ordered from low to high. FIG. 2E shows the percentages of cell subpopulations across patients, lesional and nonlesional skin (left). Note: the segments of each bar shown in the segmented bar chart represent the following marker genes from top to bottom: Basal 1, Basel 2, Cycling, B2S 1, B2S 2, Spinous, S2G 1, S2G 2, Granular, Stress 1, Stress 2, Melanocytes, DC, and TC. Comparison of the percentages of each cell subpopulation across lesional and nonlesional skin (middle). Comparison of the percentages of major cell types including keratinocytes, stress keratinocytes, melanocytes and immune cells across lesional and nonlesional skin (right). The bar plot shows that the percentages of keratinocytes and melanocytes decrease, while the percentages of stress keratinocytes and immune cells increase in lesional skin compared to nonlesional skin.

FIG. 3A, FIG. 3B, FIG. 3C, FIG. 3D, FIG. 3E, FIG. 3F, FIG. 3G, FIG. 3H, FIG. 3I, FIG. 3J, and FIG. 3K shows that stress keratinocytes have elevated metabolism and are the main source of CXCL9 and CXCL10. FIG. 3A shows a heatmap of scaled expression levels of top 10 differentially expressed genes between nonlesional and lesional keratinocytes (left). Enriched Hallmark pathways of the highly expressed genes in lesional keratinocytes (right). FIG. 3B shows violin plots comparing signature scores of WNT signaling and OxPhos pathway between nonlesional and lesional skin. P-values are from two-sided Wilcox rank tests. FIG. 3C shows a heatmap of scaled expression levels of differentially expressed genes between stressed keratinocytes and other keratinocytes. FIG. 3D shows the composition of stressed keratinocytes and other keratinocytes in nonlesional and lesional skin. FIG. 3E shows the dot plot of stress associated markers in nonlesional, lesional and stressed keratinocytes. The size represents the percentage of expressing cells and colors indicate the scaled expression. FIG. 3F shows enriched Hallmark pathways of highly expressed genes in stressed keratinocytes and other keratinocytes, respectively. FIG. 3G shows violin plots of signature scores of OxPhos, Glycolysis, WNT signaling, Interferon Gamma, Interferon Alpha, and Inflammatory response across nonlesional, lesional, and stressed keratinocytes. FIG. 3H shows an enrichment analysis of 21 metabolic pathways in stress keratinocytes vs other keratinocytes. Each dot represents one pathway. The X-axis is the differential gene signature scores of each metabolic pathway between stressed keratin and other keratinocytes. The Y-axis is Pearson's correlation of gene signature scores between each metabolic pathway and stress response. Gene signature scores of stress response were computed based on the marker genes of stressed keratinocytes. Colors (from cool to warm) represent the P-values from two-sided Wilcox rank tests of each gene signature score between stressed keratin and other keratinocytes. Of the labeled metabolic pathways: Oxidative Phosphorylation and Sphingolipid metabolism are represented by warmer tone circles, and a cooler tone circle represents Glycolysis. FIG. 3I shows the number of differentially expressed OxPhos and Glycolysis-related genes in stress keratinocytes vs. other keratinocytes. FIG. 3J shows a heatmap of the average expression of top 18 differentially expressed OxPhos-related genes between stressed keratin and other keratinocytes. The top green bars represent the difference in the proportion of expressed cells between stressed keratin and other keratinocytes. FIG. 3K shows RNAscope demonstrating Krt6A, Krt10 in situ hybridization in patient matched lesional and nonlesional punch grafting tissue. DAPI was used to stain nuclei and second harmonic generation demonstrating location of collagen.

FIG. 4A, FIG. 4B, FIG. 4C, FIG. 4D, FIG. 4E and FIG. 4F show cell-cell communication analysis reveals major signaling changes between nonlesional and lesional vitiligo skin. FIG. 4A shows the number of inferred interactions among all cell subpopulations between nonlesional (NL) and lesional (LS) skin. FIG. 4B shows the relative information flow of all significant signaling pathways within the inferred networks between nonlesional and lesional skin. Signaling pathways labeled with a square represent pathways enriched in nonlesional skin, the middle ones colored by black are equally enriched in both nonlesional and lesional skin, and the ones labeled with a circle are more enriched in lesional skin. FIG. 4C shows the visualization of outgoing and incoming interaction strength of each cell subpopulation in the inferred cell-cell communication network of nonlesional (top) and lesional skin (bottom). The dot sizes are proportional to the number of total interactions associated with each cell subpopulation. Dashed circle indicates the most altered cell subpopulations when comparing the outgoing and incoming interaction strength between nonlesional and lesional skin. FIG. 4D shows the signaling changes associated with the three most altered cell subpopulations. FIG. 4E shows a bubble plot in the left panel shows the decreased signaling from keratinocyte and immune subpopulations to melanocytes (nonlesional vs. lesional skin). Bubble plot in the right panel shows all significant signaling from stress keratinocyte to melanocytes and immune subpopulations. FIG. 4F shows inferred cell-cell communication networks of WNT and CXCL signaling in nonlesional and lesional skin, respectively (left). The edge width is proportional to the inferred communication probabilities. The relative contribution of each ligand-receptor pair to the overall signaling pathways (right).

FIG. 5A, FIG. 5B, FIG. 5C. FIG. 5D, FIG. 5E, FIG. 5F, and FIG. 5G show pseudotemporal dynamics revealing transition dynamics of stress keratinocytes. FIG. 5A shows the projection of keratinocytes onto the PHATE space, revealing the potential lineage relationships between different keratinocyte subpopulations in nonlesional (NL, left panel) and lesional (LS, right panel) skin. Cells were colored by the annotated cell identity. FIG. 5B shows the inferred pseudotemporal trajectories of all cells using Monocle 3. Cells were colored by the inferred pseudotime. Pseudotemporal trajectory analysis revealed two potential transitional paths, as indicated by Path 1 and Path 2. FIG. 5C shows pseudotemporal dynamics of all pseudo time-dependent genes along Path 1 and Path 2. Each row (i.e. gene) is normalized to its peak value along the pseudotime. These genes were clustered into five groups with average expression patterns (middle) and representative genes (right). Solid and dashed lines indicate the average expression of a particular gene group in Path 1 and Path 2, respectively. The number of genes in each gene group is indicated in parenthesis. FIG. 5D show enriched biological processes of the five gene groups in (FIG. 5C). FIG. 5E shows the reconstructed pseudotemporal dynamics of selected marker genes along the inferred pseudotime in Path 1 and Path 2, respectively. Black lines represent the average temporal patterns that were obtained by fitting a cubic spline. Cells were colored by the inferred pseudotime. FIGS. 5F and 5G show pseudotemporal dynamics of the pseudotime-dependent genes related with the stress response and OxPhos along the inferred pseudotime in Path 1 and Path 2, respectively.

FIG. 6A, FIG. 6B, and FIG. 6C show in vivo MPM imaging of vitiligo patients undergoing punch grafting and phototherapy demonstrate that metabolic changes persist in clinical non-responders. FIG. 6A shows representative clinical pictures of patients before and after punch grafting and narrowband ultraviolet B therapy in responders (top) and non-responders (bottom). FIG. 6B shows the distribution of the median β values in nonlesional and lesional skin of vitiligo patients in clinical responders (top) and non-responders (bottom); each value corresponds to a z-stack of images acquired in nonlesional and lesional skin. *=t-test p-value <0.05. FIG. 6C shows spline fits of average mitochondrial clustering (B) values based on z-stacks from lesional skin of vitiligo patients as a function of depth for responders (top) and nonresponders (bottom). Error bars represent the standard deviation of the measurements for the images in the z-stacks at each area.

FIG. 7 shows a model of stable vitiligo as an active disease state Stress keratinocytes, which exhibit a preference for oxidative phosphorylation, are enriched in vitiligo skin and secrete CXCL9 and 10 to communicate with T cells to form local inflammatory circuits. They express lower levels of Wnt ligands, which have been shown to play a role in melanocyte migration.

FIG. 8 shows Mitochondrial Clustering Distributions for Basal Keratinocytes. Mitochondrial clustering values were calculated for individually segmented cells from basal optical sections of vitiligo patients. The distributions were acquired by analyzing 182 cells from lesional regions and 258 cells from non-lesional regions. Counts were normalized to the corresponding cell totals.

FIG. 9 shows quality control metrics of the scRNA-seq data, e.g., violin plots of the number of expressed genes (nFeature_RNA), number of detected counts (nCount_RNA) and percentage of mitochondrial genes (percent mt) across all patients

FIG. 10 shows the difference between DC and TC showing a bar plot of enrichment scores (−log 10(p-value)) of enriched human cell types inferred by Enrichr (https://maayanlab.cloud/Enrichr/).

FIG. 11 shows a UMAP plot of the results of scRNA-seq data of normal skin with cell clusters labeled.

FIG. 12 shows feature plots showing expression of the stressed keratinocytes markers in the UMAP plot.

FIG. 13A, FIG. 13B, and FIG. 13C shows the pseudotime analysis results of scRNA-seq data of all patients. FIG. 13A shows marker genes' expression levels change across PAPG graph. FIG. 13B shows PAPG graph of the PHATE space. FIG. 13C shows RNA velocity across PHATE space.

FIG. 14 shows the clinical characteristics of Stable Vitiligo Patients for MPM Imaging and RNAscope

FIG. 15 shows the clinical characteristics of Stable Vitiligo Patients for scRNA-seq. The number of cells after quality control are shown.

### DETAILED DESCRIPTION OF THE INVENTION

The present invention features methods and compositions for treating pigmentation conditions such as vitiligo, e.g., inducing melanocyte repopulation, preventing further loss of melanocytes, reducing itching or burning feelings on the skin, etc. The methods feature the topical application of compositions comprising an agent that induces glycolysis for treating vitiligo. A non-limiting example of agents that induce glycolysis is biguanides. Biguanides may include but are not limited to metformin and phenformin.

As used herein, “active vitiligo” is characterized by clinical signs of confetti, trichome or koebnerization, whereas “stable vitiligo” is characterized by lesions that have not changed in more than 12 months.

The present invention also includes methods for applying glycolysis-inducing agents (e.g., biguanides) to dissociated skin cells (melanocytes and keratinocytes) during melanocyte transplantation therapy. For example, melanocytes and keratinocytes may be harvested from the skin, treated with the glycolysis-inducing agent (e.g., biguanide), and transplanted onto the patient.

Although Takano et al (Int. J. Mol. Sci. 2020, 21, 1451) showed that phenformin has a skin darkening effect, this was due to its effect on existing melanosomes (in melanocytes). Vitiligo is a condition caused by the loss of melanocytes. Thus, it is surprising that phenformin may be used to repigment skin without melanocytes in individuals with vitiligo. In Kumar Bubna (Indian J Pharmacol. 2016, 48 (1): 4-10), metformin was shown to lighten skin in hyperpigmentary disorders. Thus, it is surprising that metformin may be used to repigment (e.g., darken) skin in patients with vitiligo.

As an example, the present invention provides a method of treating vitiligo in a patient in need thereof. In certain embodiments, the method comprises topically administering a composition to at least a portion of normal skin of the patient affected by vitiligo, wherein the composition of normal skin induces glycolysis inducers and stimulates repigmentation in at least a portion of skin cells administered the composition.

The present invention also provides a method of inducing repigmentation in skin lacking melanocytes of a patient. In certain embodiments, the method comprises topically administering a composition to at least a portion of skin of the patient, wherein the composition induces glycolysis and stimulates repigmentation in at least a portion of skin cells administered the composition.

The present invention also provides a method of preventing further loss of pigmentation in a patient having vitiligo. In certain embodiments, the method comprises topically administering a composition to at least a portion of skin of the patient, wherein the composition induces glycolysis inducers and prevents further loss of pigmentation in the portion of skin administered the composition.

The present invention also provides a method of reducing symptoms of vitiligo. In certain embodiments, the method comprises topically administering a composition to at least a portion of skin of the patient, wherein the composition induces glycolysis inducers and reduces symptoms in the portion of skin administered the composition. Symptoms may include itching and/or a burning sensation.

Referring to any of the methods herein, the composition may comprise a biguanide such as but not limited to metformin, phenformin, the like, or a combination thereof.

In certain embodiments, the composition further comprises a pharmaceutically acceptable carrier. Pharmaceutically acceptable carriers, particularly those for topical application of medicaments or compositions, are well known to one of ordinary skill in the art.

In certain embodiments, the method further comprises co-administering a secondary therapeutic. A non-limiting example of a secondary therapeutic is a JAK inhibitor. In certain embodiments, the secondary therapeutic is administered at the same time as the composition. In certain embodiments, the secondary therapeutic is administered at a time before the composition is administered. In certain embodiments, the secondary therapeutic is administered at a time after the composition is administered.

The present invention also features a composition for use in a method of treating vitiligo, a method of inducing repigmentation in skin lacking melanocytes, a method of preventing further loss of pigmentation in a patient having vitiligo, and/or a method of reducing symptoms of vitiligo. In some embodiments, the method comprises topically administering a composition to at least a portion of skin of the patient affected by vitiligo. Referring to the composition, the composition may comprise a glycolysis-inducing agent that stimulates glycolysis in skin cells having been contacted with the composition.

In some embodiments, the glycolysis-inducing agent is a biguanide. In some embodiments, the biguanide is metformin. In some embodiments, the biguanide is phenformin.

In some embodiments, the composition further comprises a pharmaceutically acceptable carrier. In some embodiments, the composition further comprises a secondary therapy. In some embodiments, the secondary therapy is a JAK inhibitor.

The present invention also features a method of preparing a population of melanocytes and keratinocytes. In some embodiments, the method comprises harvesting melanocytes and keratinocytes from a portion of skin in a patient; and subjecting the melanocyte keratinocyte mixture to a treatment, said treatment comprising a composition comprising a glycolysis-inducing agent, e.g. a biguanide

The present invention also features a method of transplanting melanocytes and keratinocytes. In some embodiments, the method comprises harvesting melanocytes and keratinocytes from a portion of skin in a patient; subjecting these skin cells to a treatment, said treatment comprising a composition comprising a glycolysis-inducing agent (e.g., a biguanide); and transplanting the melanocytes onto the patient.

### Example

The following is a non-limiting example of the present invention. It is to be understood that said example is not intended to limit the present invention in any way. Equivalents or substitutes are within the scope of the present invention.

Vitiligo is an autoimmune skin disease characterized by the progressive destruction of melanocytes by autoreactive CD8⁺ T cells, resulting in disfiguring patches of white depigmented skin that cause significant psychological distress among patients. CD8⁺ T cells play an important role in eliminating melanocytes and are increased in active vitiligo skin. However, in stable vitiligo lesions devoid of melanocytes, T cells are sparse and immune activation levels are low. This makes it unclear why white patches continue to persist in the absence of a robust inflammatory infiltrate. The development of mouse models representative of human disease has provided important clues on the role of the adaptive immune system in vitiligo. Keratinocytes secrete CXCL9 and CXCL10 to attract and activate CXCR3⁺ CD8⁺ T cells, and these chemokines are present in the blister fluid of human vitiligo patients. However, the adoptive transfer of autoreactive CD8⁺ T cells in the mouse model cannot fully recapitulate the complex interactions between melanocytes, keratinocytes, and immune cells that occurs in situ in human skin-melanocytes are present in the epidermis in only select locations in mice and the mouse epidermis is considerably thinner and lacks the stratification seen in human skin. To date, most translational studies in vitiligo are limited to examining cultured cells in vitro or immunohistochemistry of diseased tissue. It has been challenging to study how cell lineages collectively contribute to disease persistence, secondary to the lack of tools to assess cellular heterogeneity in vivo. Multiphoton microscopy (MPM) is a unique tool for this purpose and has broad applications in human skin. MPM is a noninvasive imaging technique capable of providing images with sub-micron resolution and label-free molecular contrast which can be used to characterize keratinocyte metabolism in human skin. This approach is based on the two-photon excited fluorescence (TPEF) signal detected from the reduced nicotinamide adenine dinucleotide (NADH), a coenzyme in the keratinocyte cytoplasm that plays a central role in metabolism. This technique's ability to assess cellular metabolism has been validated in normal skin under hypoxic conditions. Further addition of multiparametric analyses of NADH and flavin adenine dinucleotide (FAD) allows for quantification of metabolic changes at a single cell resolution in cells and tissue.

The present invention employs MPM for in vivo imaging of stable vitiligo lesions and assesses keratinocyte metabolic state based on an imaging metric derived from a mitochondrial clustering analysis approach previously validated. Single-cell RNA sequencing (scRNA-seq) is then performed on patient-matched lesional and nonlesional tissue to identify keratinocyte subpopulations that express chemokines known to drive vitiligo pathogenesis. By applying CellChat, a tool that quantitatively infers and analyzes intercellular communication networks in scRNA-seq data, it was determined that stress keratinocytes communicate with adaptive immune cells via the CXCL9/10/CXCR3 axis to create local inflammatory loops that are active in stable vitiligo. Moreover, signaling between melanocytes and keratinocytes via the WNT pathway was altered in stable vitiligo lesions. By integrating non-invasive MPM, scRNAseq, and advanced bioinformatics, communication networks were inferred between keratinocytes, melanocytes, and immune cells capable of preventing normal melanocyte repopulation

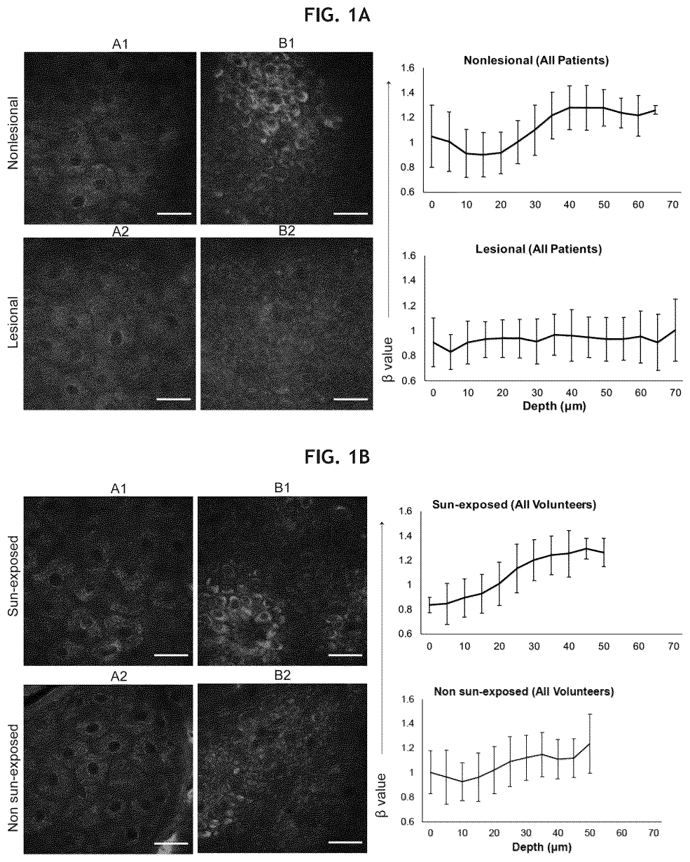

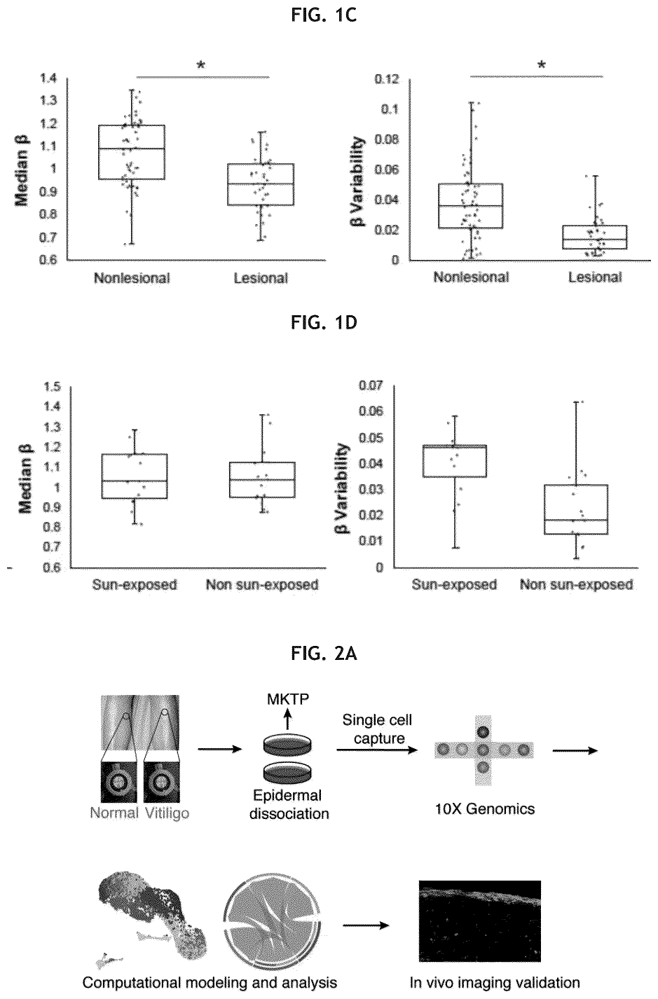

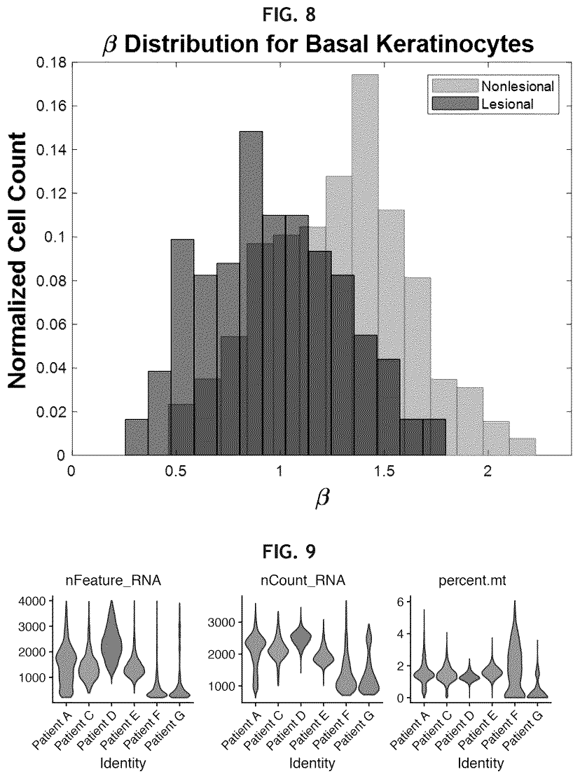

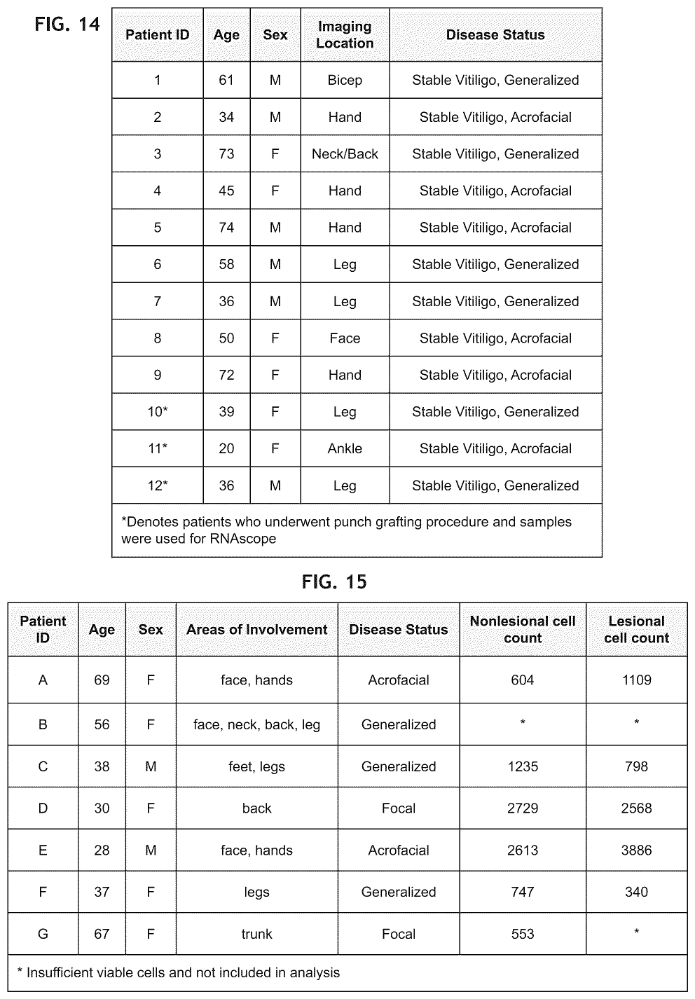

MPM imaging of stable vitiligo skin in vivo demonstrates mitochondrial clustering changes: To look at epidermal changes using MPM in stable vitiligo, the MPTflex clinical microscope was utilized to image twelve patients with lesions characterized by depigmented areas that have not grown in size for at least one year and did not exhibit active vitiligo features such as confetti-like depigmentation, koebnerization and trichome (FIG. 14). As expected, MPM images of nonlesional skin showed brighter fluorescence spots in the cellular cytoplasm, which represent aggregates of melanosomes, compared to lesional skin (FIG. 1A). Nonlesional skin exhibits depth-dependent changes in mitochondrial clustering that reflects differences in metabolism (FIG. 1A) In short, the basal and parabasal keratinocytes present a fragmented mitochondria phenotype characterized by high values of the mitochondrial clustering metric, β. As cell differentiation progresses from the basal to the higher epidermal layers and cells turn from glycolysis to oxidative phosphorylation for energy production, mitochondria fuse and create more extensive networks that correspond to lower clustering values, reaching their minima within the spinous layer (FIG. 1A). Finally, toward the most terminally differentiated layer, as the granular keratinocytes enter an apoptotic state to create the stratum corneum, mitochondrial clustering values recover again, signifying a return to a more fissioned phenotype. In contrast, lesional depigmented skin from vitiligo patients showed an altered trend of mitochondrial clustering compared to nonlesional skin (FIG. 1A), suggesting that the depth-dependent metabolic changes were lost. The mitochondrial clustering (B) median value and its variability were calculated across the epidermis of vitiligo and normal skin and found that these metrics are significantly different in vitiligo lesional and nonlesional skin (FIG. 1C). Given that these changes were observed in the basal layer, additional analysis was performed to compare mitochondrial clustering between lesional and nonlesional basal keratinocytes. This analysis indicates a more heterogeneous distribution of mitochondrial clustering. B, values for lesional vitiligo vs. non-lesional basal keratinocytes (FIG. 8), yielding distributions with heterogeneity index values of 0.16 and 0.12, respectively. Noticeably, vitiligo basal keratinocytes exhibited an increase in the number of cells characterized by lower mitochondrial fragmentation levels and, thus, more networked mitochondria, consistent with enhanced oxidative phosphorylation.

Since the fluorescence signals from all the skin fluorophores, including NADH, are collected on the same detection channel in the MPTflex, thus, tests were done to ensure the mitochondrial clustering measurements were not affected by contributions from fluorophores other than NADH. Melanin requires particular consideration since it is the primary source of the difference in appearance between vitiligo and normal skin Mitochondrial clustering was measured in five healthy volunteers to ensure that melanin content was not affecting fluorescence signals. Melanin content was controlled for by comparing sun-exposed sites (dorsal forearm) and non sun-exposed sites (volar upper arm, which would have relatively less melanin). Depth-dependent B values showed similar trends in the epidermis (FIG. 1B) regardless of sun-exposure status, and the median B values and B variability values were not significantly different (FIG. 1C). These results confirmed that mitochondrial clustering in basal and parabasal keratinocytes of lesional skin was altered compared to nonlesional skin. This was a result of changes to the mitochondrial organization in vitiligo skin and was not a consequence of differences in melanin content.

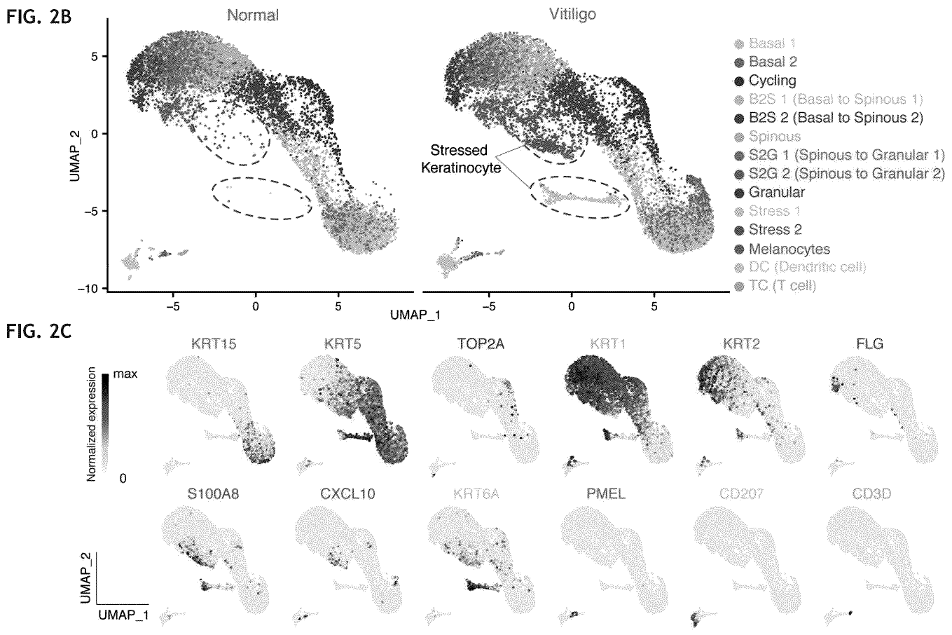

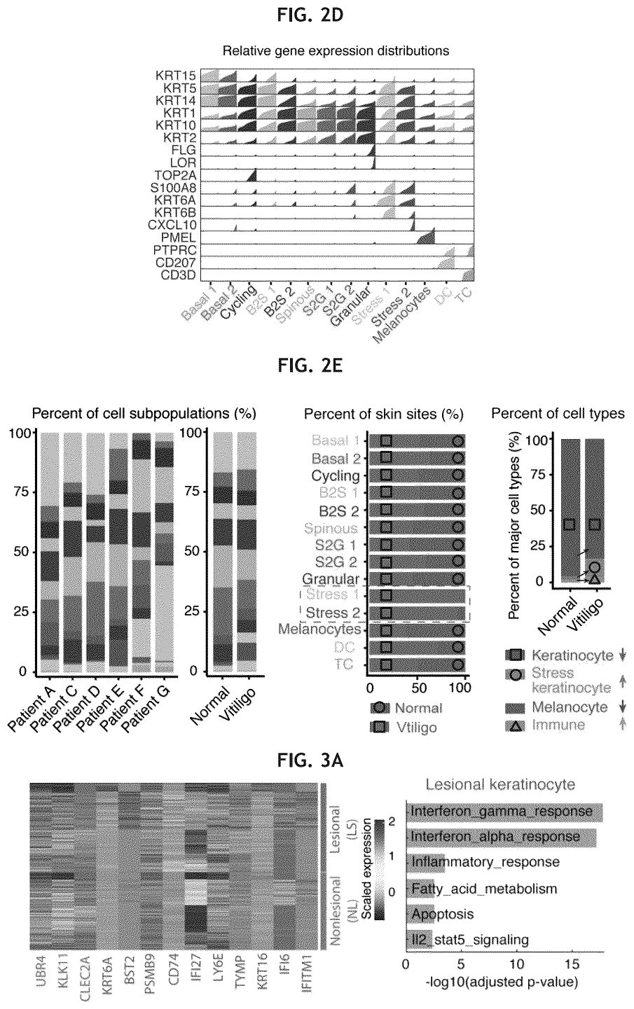

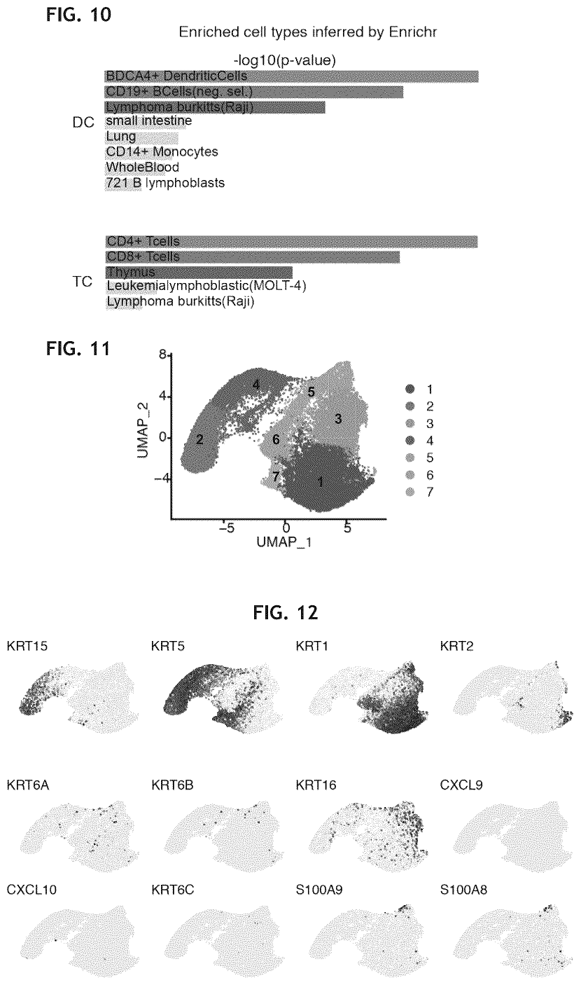

scRNA-seq reveals unique keratinocyte cell states enriched in vitiligo lesional skin: MPM imaging demonstrated that basal and parabasal keratinocytes in vitiligo lesions were metabolically altered, suggesting that keratinocyte cell states are different in vitiligo patients. To systematically examine the major keratinocyte cell state changes in vitiligo, scRNAseq was performed on a separate group of patient-matched lesional and nonlesional suction blisters from seven patients using the 10× Genomics Chromium platform (FIG. 2A). 1 set of samples (patient B) was excluded from further analyses due to the low viability of cells (FIG. 15). Read depth normalization and quality control was performed (FIG. 9), and a total of 9254 cells of vitiligo lesional skin and 7928 cells of nonlesional skin were obtained for downstream analyses. Integration analysis of data was performed from all patients using a recently developed approach, scMC, designed to preserve biological signals while removing batch effects. Unsupervised clustering analysis identified 14 cell clusters (FIG. 2B). Clusters were attributed to their putative identities using the differentially expressed gene signatures (FIG. 2C, FIG. 2D), including basal keratinocytes (high KRT15 and KRT5 expression), spinous keratinocytes (high KRT1 expression), granular keratinocytes (high FLG and LOR expression), cycling keratinocytes (high TOP2A expression), melanocytes (high PMEL expression), TC (T cell) (high CD3D expression) and DC (Dendritic cell) (high CD207 expression) (FIG. 2B). The intermediate keratinocyte states, including basal to spinous transition and spinous to granular transition, were defined based on the hybrid expression of KRT15, KRT1, and KRT2. Notably, two keratinocyte states were identified that upregulate expression of keratins that are not generally expressed in the mature interfollicular epidermis and are associated with insults like wounding and UV injury (FIG. 2B). Stress 1 subpopulation was highly enriched for KRT6A while Stress 2 subpopulation expressed KRT6A at lower levels. They also expressed KRT16 and S100A8/9, alarmins associated with local inflammation that have been used as biomarkers for other inflammatory conditions. These populations were termed “stress keratinocytes” as their transcriptional signature corresponds with injuries and inflammation. Interestingly, stress keratinocytes were only enriched in vitiligo lesional skin (FIG. 2B). Detailed analysis of the two immune cell subpopulations TC and DC showed that they were distinguished from each other with clearly distinct gene signatures and biological processes (FIG. 10). Cellular composition analysis showed that although different patients exhibited certain heterogeneity, cell clusters were common amongst patients (FIG. 2E). Compared to nonlesional skin, vitiligo lesional skin showed dramatically increased presence of stress keratinocyte and to a lesser extent of DC, and a clear decrease of melanocytes (FIG. 2E). Overall, the percentages of keratinocytes and melanocytes were decreased, and stress keratinocytes and immune cells were increased in vitiligo lesional skin (FIG. 2F). Moreover, keratinocytes were analyzed from normal human skin using a previously published scRNA-seq dataset (FIG. 11, FIG. 12), but did not observe the expression of stress signature genes, suggesting that stress keratinocytes were uniquely enriched in vitiligo lesional skin. Integration analysis using a Seurat package produced similar cellular compositions but did not preserve biological variation as well. In particular, stress keratinocytes were intermixed with other keratinocyte cell states and were in a spread distribution in the UMAP space (Uniform Manifold Approximation and Projection) (data not shown).

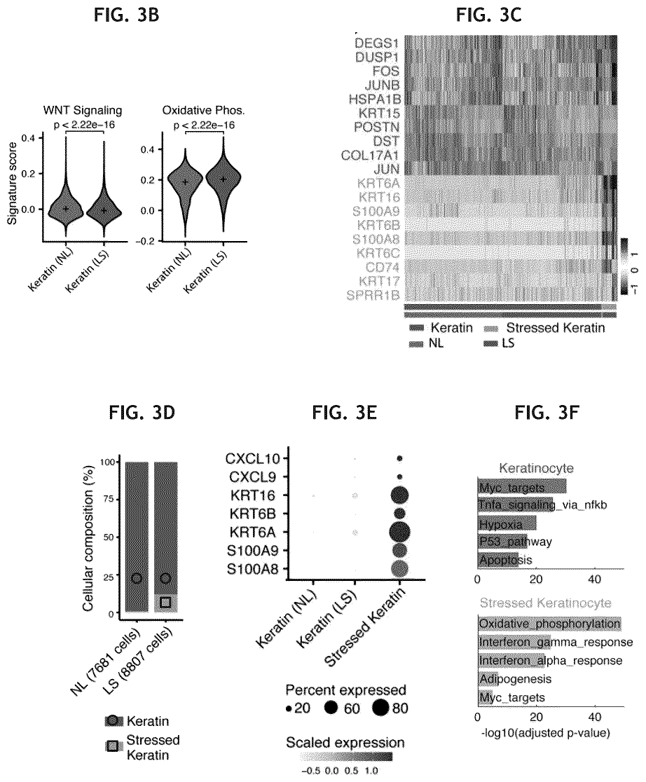

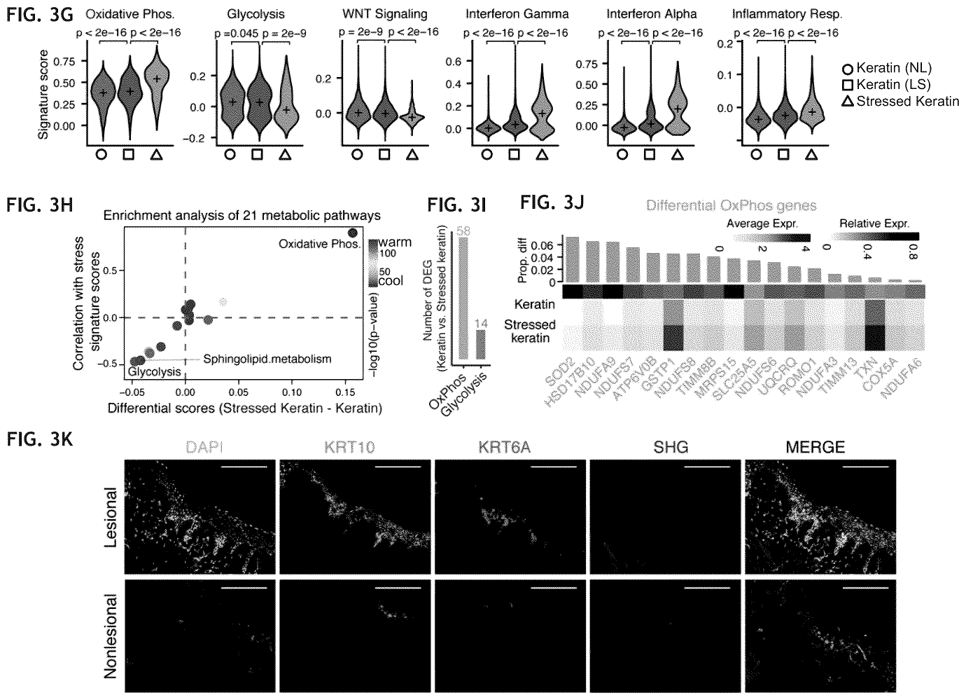

Stress keratinocytes exhibit altered metabolism with dominant upregulation of OxPhos: To further characterize keratinocyte differences in detail between vitiligo lesional and nonlesional skin, differential expression analysis was first performed and found that lesional skin expressed higher levels of KRT6A and KRT16 keratins that are not generally expressed in the mature interfollicular epidermis and are associated with insults like wounding and UV injury (FIG. 3A). Inflammatory and immune response related genes such as CD74, IFI27, IFI6 and IFITM1 were also significantly increased, which was further confirmed by the hallmark pathway enrichment analysis of the genes highly expressed in vitiligo lesional skin using the Molecular Signatures Database (MSigDB. FIG. 3A). In addition, the top two enriched pathways were interferon-gamma and alpha responses (FIG. 3A), which is consistent with previous findings that lesional keratinocytes differed from their nonlesional counterparts in upregulation of interferon responses (FIG. 3A). Gene scoring analysis revealed downregulation of WNT signaling (FIG. 3B), consistent with the known role of WNT in melanocyte pigmentation. Since MPM demonstrated metabolic differences between nonlesional and lesional vitiligo skin, the signature scores of oxidative phosphorylation (OxPhos) were further computed. Interestingly, higher scores were observed in lesional skin (FIG. 3B).

The difference between keratinocytes and stress keratinocytes and stress keratinocytes was next focused on to determine whether the above observed differences in signaling and metabolism were attributed to the unique stress keratinocytes in vitiligo lesional skin. Differential expression analysis revealed distinct gene signatures between these two keratinocyte states (FIG. 3C and FIG. 3F). In addition to KRT6, KRT16, KRT17. S100A8, and 9 alarmins are expressed in stress keratinocytes (FIG. 3C). Hallmark gene enrichment analysis of the differentially expressed genes showed that stress keratinocytes were enriched by OxPhos and interferon responses (FIG. 3F). Since there were nearly no stress keratinocytes in nonlesional skin (FIG. 3D), three keratinocyte groups were focused on: nonlesional keratinocytes, lesional keratinocytes, and lesional stress keratinocytes. Comparison of these groups showed that CXCL9/10, KRT16, KRT6A/B, and S100A8/9 were specifically expressed in stress keratinocytes instead of the other two keratinocyte groups (FIG. 3E). Further quantitative comparison was performed on these three keratinocyte groups using gene scoring analysis Impressively, dramatic differences between stress keratinocytes and both lesional and nonlesional keratinocytes, in terms of OxPhos, Glycolysis, WNT signaling. Interferon Gamma, Interferon Alpha and Inflammatory response (FIG. 3F). Notably, significantly increased OxPhos and decreased glycolysis were consistent with the MPM imaging data (FIG. 3F and FIG. 1A). These results suggest that stress keratinocytes in vitiligo lesional skin dominantly account for the observed differences in signaling and metabolism between lesional and nonlesional skin.

To further examine whether OxPhos and glycolysis were the prominently impaired metabolic processes in vitiligo lesional skin, the enrichment of 21 metabolic pathways was quantitatively evaluated using gene scoring analysis. OxPhos and Glycolysis were the most significantly altered pathways among all 21 metabolic pathways, which showed the largest differences between stress keratinocytes and other keratinocytes and the strongest correlations with stress signatures (FIG. 3G). Of note, OxPhos and Glycolysis were highly positively and negatively correlated with stress signatures, respectively. There are 58 and 14 differently expressed OxPhos and Glycolysis related genes between stress keratinocytes and other keratinocytes (FIG. 3H). Stress keratinocytes were enriched for genes associated with OxPhos, including SOD2, NDUFA9, and ATP6VOB. In contrast, keratinocytes expressed higher levels of genes associated with Glycolysis, including ALDH3A2, SDC1 and HSPA5. These results, combined with MPM data, indicate that a subpopulation of cells in vitiligo skin have altered metabolism and upregulate of OxPhos. RNAscope was then performed on patient-matched lesional and nonlesional skin to localize this keratinocyte population using KRT6A as it is highly expressed in this population (FIG. 2C). consistent with the MPM imaging. KRT6A expressing cells were enriched in the basal layer of the epidermis (FIG. 3K).

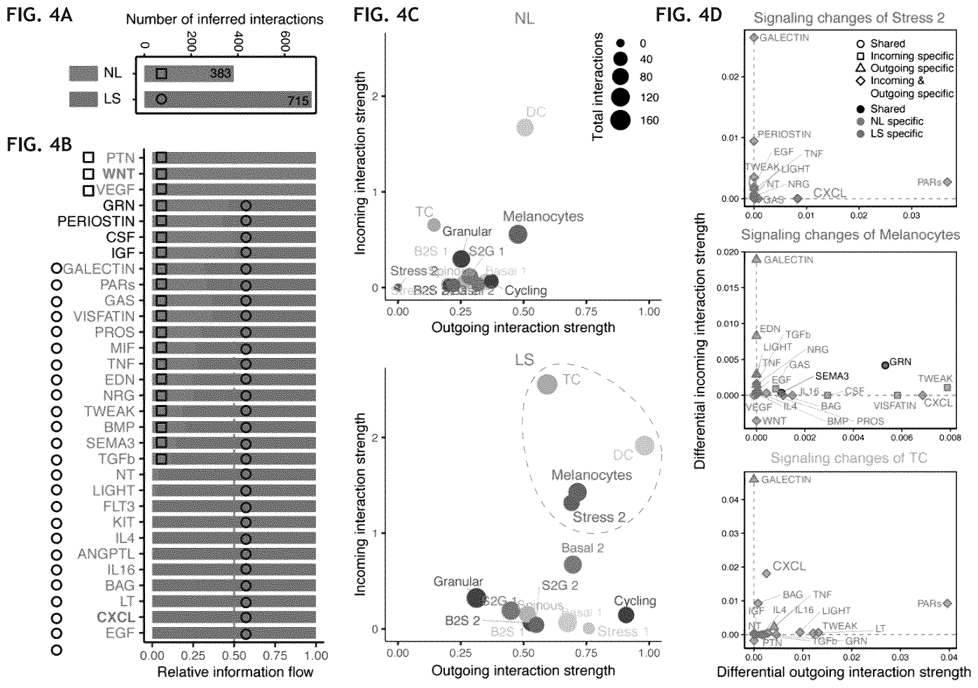

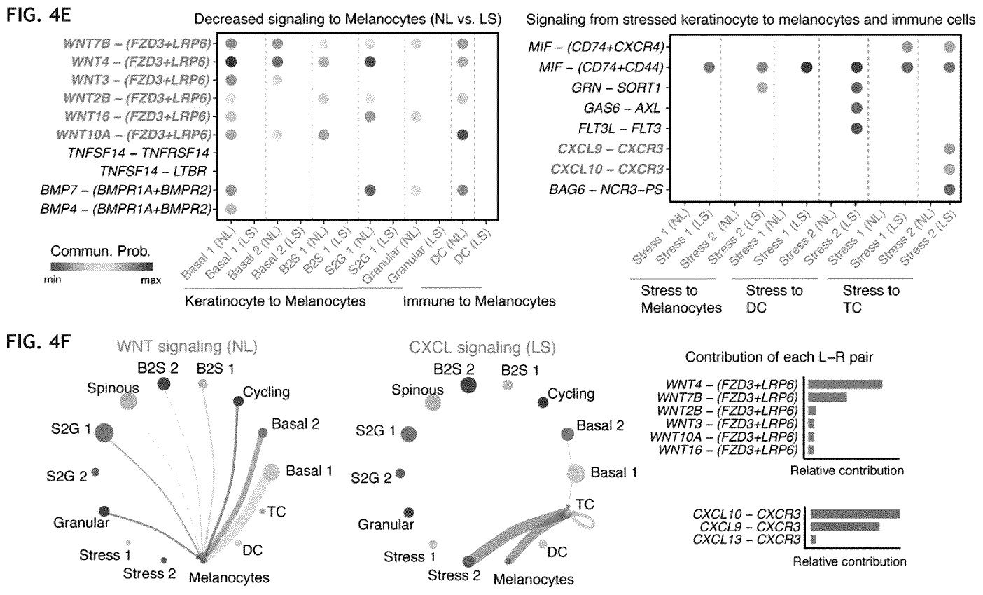

Analysis of cell-cell communication reveals major signaling changes in response to vitiligo: To systematically detect major signaling changes in stable vitiligo lesions, a recently developed tool, CellChat, was applied to the scRNA-seq data of both nonlesional and lesional skin. Twice the number of interactions in lesional skin were observed compared to nonlesional skin (FIG. 4A). Each signaling pathway was compared between nonlesional and lesional skin using the concept of information flow defined as a sum of the communication probability among all pairs of cell groups, to study the prominent signaling pathways that contribute to the dysfunctional signaling in lesional skin. Several pathways were only activated in nonlesional skin (FIG. 4B), including WNT, PTN, and VEGF, consistent with the role of WNT activation in regulating melanocyte differentiation. In contrast, many inflammatory pathways prominently increase their information flow at lesional skin as compared to nonlesional skin, such as CXCL, IL4, IL6, LT, LIGHT, TWEAK, TNF, VISFATIN and GALECTIN.

Next, how different cell subpopulations changed their signaling patterns from nonlesional to lesional skin was determined using network centrality analysis, which computes the outgoing and incoming interaction strength of each subpopulation to represent the likelihood as signaling sources and targets, respectively. This analysis revealed that T cells emerged as major signaling targets while dendritic cells (DC) became dominant signaling sources. In addition, melanocytes and Stress 2 keratinocytes also prominently increased their outgoing and incoming signaling from nonlesional to lesional skin (FIG. 4C). Differential interaction analysis showed that the prominently increased outgoing signaling of Stress 2 keratinocytes and Melanocytes and the incoming signaling to T cells were CXCL (FIG. 4D), suggesting that CXCL signaling pathway was the dominantly dysfunctional signaling sent from Stress 2 keratinocytes and Melanocytes to T cells. Of note, WNT is the major decreased incoming signaling of Melanocytes.

By studying the signals sent to melanocytes, it was found that a relative deficiency of WNT and BMP signaling was noted in keratinocytes and DC in lesional skin. In particular, the WNT signal was seen in all keratinocyte populations in nonlesional skin with WNT4 and WNT7B driving the signaling (FIG. 4E, FIG. 4F). For the signaling from stress keratinocyte to melanocytes, DC and TC cells, MIF and CXCL signaling were highly active in lesional skin. Notably, for the signaling from stress keratinocyte to TC, ligands CXCL9 and CXCL10 and their receptor CXCR3 were found to be uniquely active in lesional skin (FIG. 4E, FIG. 4F). Taken together, these analyses indicated the prominent alteration of cell-cell communication networks in vitiligo lesional skin and predicted major signaling changes that might drive vitiligo pathogenesis.

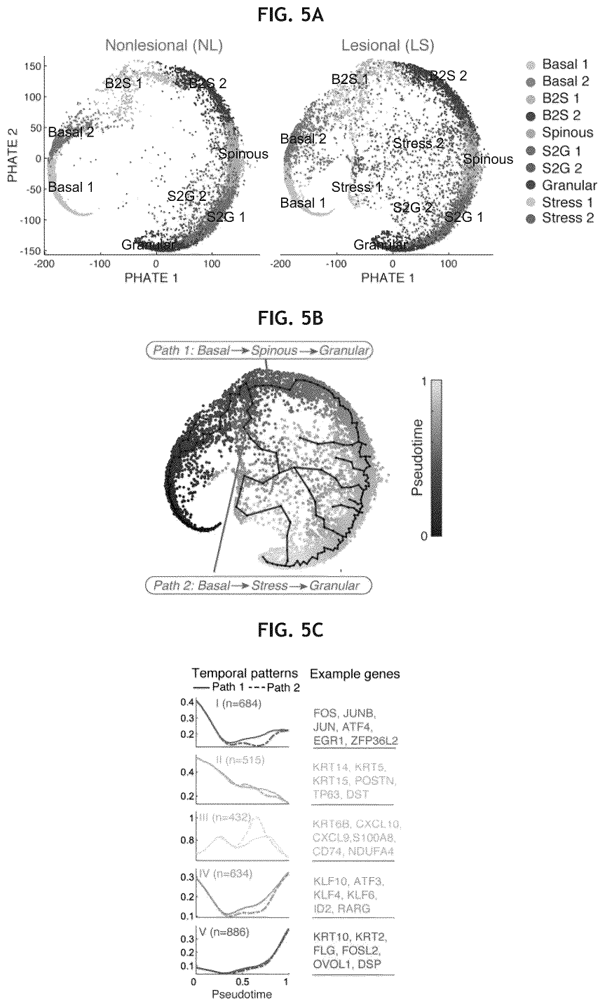

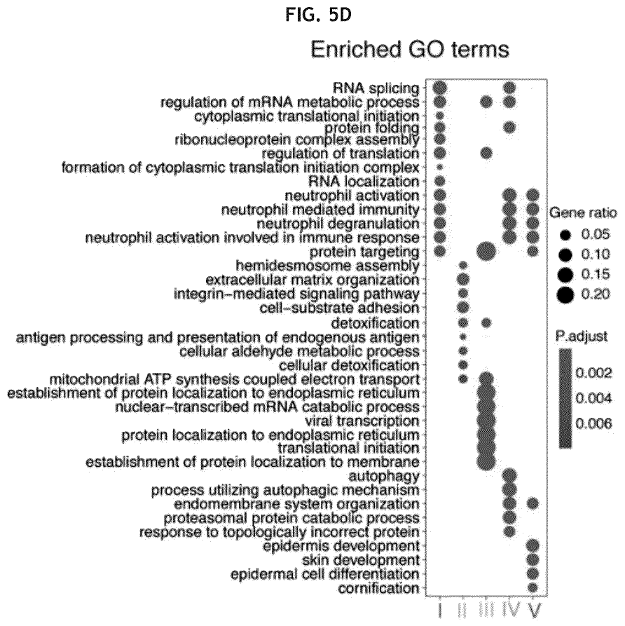

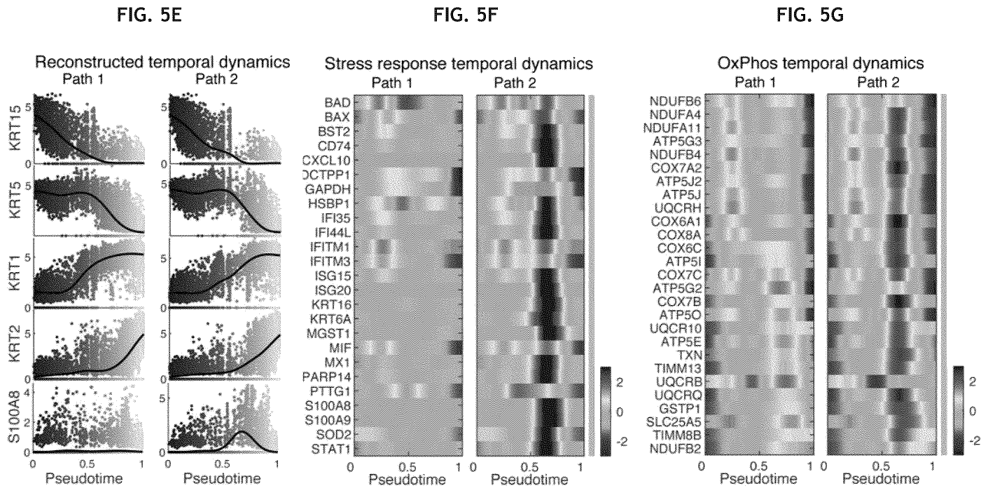

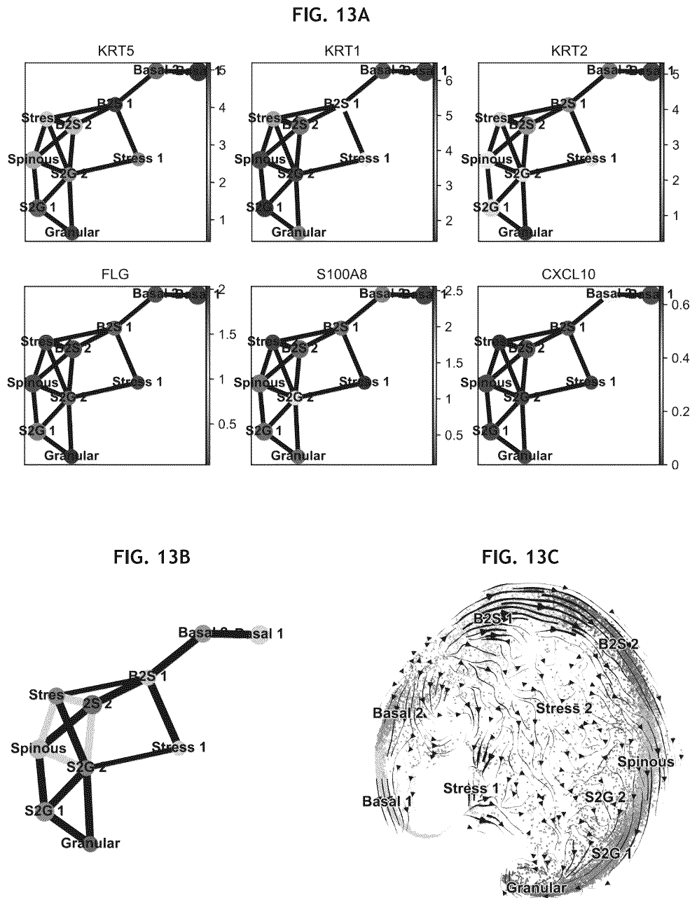

Pseudotemporal dynamics reveal transition dynamics of stress keratinocytes: Pseudotemporal trajectory analysis was performed using all keratinocyte cells except for cycling cells from all samples to explore the role of stress keratinocytes in keratinocyte differentiation. By applying the diffusion-based manifold learning method PHATE to the batch corrected data obtained from scMC, a differentiation path was observed in the nonlesional skin, recapitulating sequential stages of keratinocyte differentiation process from basal state to terminally differentiated granular state. However, in vitiligo lesional skin, in addition to the known keratinocyte differentiation path (Path 1), another potential differentiation path (Path 2) was found to contribute to stress keratinocytes (FIG. 5A). Using an unsupervised pseudotemporal trajectory inference tool Monocle 3, the stress keratinocytes indeed contributed to alternative differentiation paths, indicating a transition from an early intermediate keratinocyte state (basal to spinous transition) to stress keratinocytes, to a late intermediate keratinocyte state (spinous to granular transition), and then to granular state (FIG. 5B). Such observation was further confirmed using another trajectory inference approach PAGA, showing strong likelihood of the transition between stress keratinocytes and the late keratinocyte states (FIG. 13A). To further analyze the keratinocyte differentiation dynamics, RNA velocity analysis was performed using scVelo, a computational tool that can predict potential directionality and speed of cell state transitions based on levels of spliced and unspliced mRNA. RNA velocity analysis also provided evidence for enhanced transition dynamics from stress keratinocytes to the late keratinocyte state (FIG. 13B). Together, in addition to the normal keratinocyte differentiation trajectory, these analyses showed the transition dynamics of stress keratinocytes contribute to an altered keratinocyte differentiation trajectory in vitiligo lesional skin.

Next key molecular changes that may be important for keratinocyte cell state transitions were identified using scEpath. scEpath identified 1284 and 3151 pseudotime-dependent genes over the normal (Path 1) and alternative keratinocyte differentiation trajectories (Path 2), respectively (FIG. 5C). These pseudotime-dependent genes were further classified into five groups based on their pseudotemporal dynamics. Interestingly, gene group III exhibited distinct expression dynamics along Path 1 versus Path 2, while the remaining gene groups followed very similar dynamical trends on both trajectories. Furthermore, genes in Group III included not only stress keratinocyte-related signatures such as KRT6B, CXL10, CXCL9, S100A8, and CD74, but also OxPhos-associated signatures such as NDUFA4 and ATP5G3 (FIG. 5C). Further GO enrichment analysis revealed distinct enriched biological processes among these five gene groups, including the enriched metabolic processes in group III (FIG. 5D). The reconstructed pseudotemporal dynamics of typical maker genes well recapitulated the expected keratinocyte differentiation dynamics (FIG. 5E). As expected, stronger activation of stress response, inflammatory response, and OxPhos associated genes was observed in the Path 2 compared to Path 1 (FIG. 5F, FIG. 5G). Taken together, stress keratinocytes induce an altered keratinocyte differentiation trajectory with strong activation of inflammatory response and OxPhos related gene expression in vitiligo lesional skin.

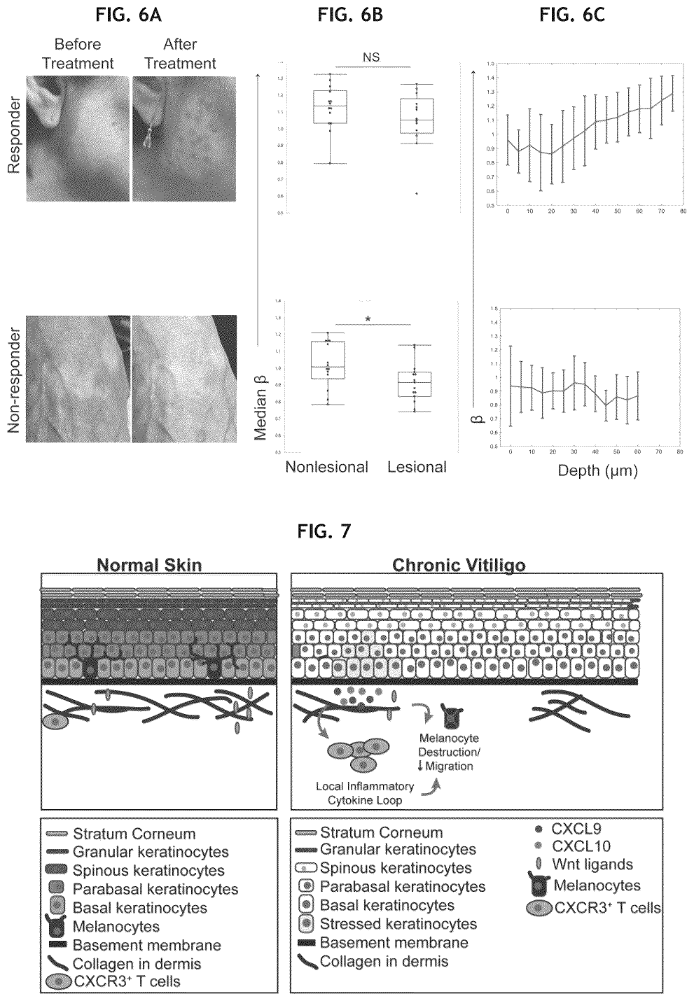

Stress keratinocytes normalize in patients that respond to punch grafting treatment: The noninvasive imaging and scRNA-seq data suggest that it is feasible to use MPM to track metabolically altered populations of keratinocytes in patients with vitiligo. Stable vitiligo patients undergoing a combination of punch grafting and phototherapy treatment were followed to determine how stress keratinocytes change by imaging their skin lesions with MPM at baseline and 10 weeks after treatment. In patients that responded to treatment and demonstrated repigmentation (FIG. 6A, top), keratinocyte mitochondrial clustering values (B) resembled nonlesional skin after treatment (FIG. 6B, top), and epidermal depth-dependent shift towards glycolysis at the basal layer was restored (FIG. 6C). In contrast, clinical non-responders (FIG. 6A, bottom) had persistent changes in mitochondrial clustering values (FIG. 6B, bottom) similar to vitiligo lesional skin at baseline (FIG. 1A). The epidermal depth dependent shift towards oxidative phosphorylation seen in baseline vitiligo lesional skin remained stable (FIG. 6C, bottom), suggesting that metabolically altered stress keratinocytes persisted in clinical non-responders. These findings suggest that the presence of metabolically stressed keratinocytes are associated with a lack of clinical response.

To date, the study of human vitiligo and cell-cell interactions in the tissue microenvironment (TME) has largely been limited to traditional in vitro cultures and immunohistochemistry methods due to the lack of tools to assess cellular changes in situ. Described herein, the combination of MPM in vivo imaging of stable vitiligo patients and various scRNA-seq analyses to demonstrate that a small subpopulation of stress keratinocytes in the basal/parabasal layer exhibit a unique signature-metabolic preferences for oxidative phosphorylation, expression of stress keratins, alarmins and CXCL9/10 and diminished WNT signaling—and likely drive the persistence of white patches in vitiligo. This data suggests that it is feasible to use MPM as a noninvasive method to track metabolically altered populations of keratinocytes in vitiligo. Previous studies on metabolic alterations in vitiligo largely focused on melanocytes' increased susceptibility to oxidative insults such as H₂O₂ due to decreased expression of antioxidant pathways. Oxidative stress led to HMGB1 release by cultured melanocytes, which then stimulated cytokine release by keratinocytes. Studies on cultured keratinocytes from vitiligo skin showed swollen mitochondria and similar increased susceptibility to oxidative stress. However, definitive studies looking at keratinocyte metabolism and its contributions to vitiligo have been lacking. The present invention addresses this gap and demonstrates that specific basal and parabasal keratinocyte states exhibit increased OXPHOS and communicate with T cells via the CXCL9/10/CXCR3 axis and exhibit decreased WNT signaling to melanocytes.

Most studies on vitiligo have focused on active disease, and the importance of the CXCL9/10/CXCR3 axis is well established from studies on human skin samples. Stable vitiligo, however, remains enigmatic. Transcription analyses on depigmented whole skin show minimal immune activation with no CXCL10 elevation. Flow cytometry of stable vitiligo skin blisters demonstrated the presence of a small population of melanocyte-specific CD8⁺ resident memory T cells (TRM), and depletion of TRM by targeting CD122 led to repigmentation in a mouse model of vitiligo. By using scRNA-seq to identify changes in cellular compositions in stable vitiligo skin, a keratinocyte state was identified with transcriptome changes important in communicating with other cell types to drive disease persistence. The signals from stress keratinocytes were likely lost from averaging cell gene expression in previous whole skin transcriptional studies, accounting for observed differences in CXCL10 expression in the present study. By utilizing CellChat analyses, the data herein highlights that in stable vitiligo, a small epidermal niche of metabolically altered stress keratinocytes communicate with T cells and melanocytes to form local inflammatory circuits to drive disease persistence (FIG. 6A, FIG. 6B, and FIG. 6C), highlighting vitiligo involves multiple etiologic factors. In patients that undergo punch grafting and phototherapy, the persistence of stress keratinocytes is associated with a lack of clinical response, further implicating the role of stress keratinocytes in chronic disease. Keratinocytes as drivers of local inflammatory loops have been suggested in atopic dermatitis and psoriasis. Similar loops are important in vitiligo persistence but how stress keratinocytes are established in the first place and whether they play a key role in the maintenance of this cellular circuitry remain obscure (FIG. 7).

The data indicate that stress keratinocytes have altered metabolic preferences, drive local inflammation in the skin microenvironment and can be visualized in situ in human patients using noninvasive MPM imaging. These results are significant because they provide evidence for a potential link between stress keratinocytes and vitiligo persistence. They also indicate that MPM imaging can also be used to follow vitiligo patients longitudinally to better understand the role stress keratinocytes play in disease pathogenesis and identify areas that could be targeted by new therapies. These new therapies could range from targeted destruction of altered keratinocytes (laser therapies) or pharmacologic modulation of their physiology.

Study Design: This study utilized noninvasive MPM and scRNA-seq to study patient-matched lesional vs. nonlesional skin in stable vitiligo and how intercellular communications are affected in depigmented skin. Imaging, suction blister and punch skin biopsy of patients were performed under IRB-approved protocols at UC Irvine and samples were de-identified before use in experiments. Vitiligo skin samples were obtained after examination by board-certified dermatologists. Control skin was acquired from tumor excision tips without notable pathology from patients without vitiligo. Stable vitiligo lesions were characterized by the absence of koebnerization, confetti-like depigmentation or trichome lesions and those that have not grown in size for at least one year. Non-lesional sites were selected as normal appearing, non-depigmented skin on the thigh when examined by Wood's lamp.

Patients for Imaging: Twelve vitiligo patients and five volunteers with normal skin were imaged in vivo by MPM. All vitiligo patients had stable vitiligo, defined by no change in size for at least one year and did not exhibit features of active vitiligo such as koebnerization, confetti-like depigmentation and trichome. Patients were previously unresponsive to past treatment attempts and had no treatment in the three months before imaging for this study. Vitiligo patient ages were 34-74 with an average age of 56. Vitiligo lesion locations included wrist, hand, leg, arm, face, and neck. Nonlesional pigmented skin was selected after Wood's lamp exam on separate body sites or at least 12 cm from the closest depigmented macule. All in vivo measurements were conducted according to an approved institutional review board protocol of the University of California, Irvine (HS No. 2018-4362), with written informed consent obtained from all patients.

MPM Imaging: An MPM-based clinical tomograph (MPTflex, JenLab, GmbH, Germany) was used for the in vivo imaging of the vitiligo and normal skin. This imaging system consists of a femtosecond laser (Mai Tai Ti: Sapphire oscillator, sub-100 fs, 80 MHz, tunable 690-1020 nm; Spectra-Physics), an articulated arm with near-infrared optics, and beam scanning module. The imaging head includes two photomultiplier tube detectors used for parallel acquisition of two-photon excited fluorescence (TPEF) and second harmonic generation (SHG) signals. The excitation wavelength used in this study was 760 nm. The TPEF and SHG signals were detected over the spectral ranges of 410 to 650 nm and of 385 to 405 nm, respectively. A Zeiss objective (40x, 1.3 numerical aperture, oil immersion) was used for focusing the laser light into the tissue. The laser power used was 5 mW at the surface and up to 30 mW in the superficial dermis of the skin. The MPM data was acquired as z stacks of en-face images from the stratum corneum to the superficial dermis. The field of view (FOV) for each optical section was 100×100 μm² and the step between the optical sections was 5 μm. The patients' vitiligo lesional area were imaged, and a normally pigmented area on the upper thigh as control. The rationale for selecting the thigh location as control site for imaging was based on the fact that the patients imaged, being unresponsive to prior treatment of vitiligo, were scheduled for micrografting therapy. Imaging locations for healthy volunteers with normal skin were the sun exposed dorsal forearm, and the non-sun exposed volar upper arm to focus on areas with relatively higher pigment amounts (sun-exposed), and relatively lower pigment amounts (non sun-exposed). Due to the limited FOV of each individual scan, several stacks of images were acquired within each site in order to sample a larger area. Thus, a total of 1,872 images were acquired for this study, corresponding to an average of 18 images for each imaging site. Images were 512×512 pixels and were acquired at approximately 6 s per frame. All images were color-coded such that green and blue represent the TPEF and SHG signals, respectively. In MPM imaging of skin, the contrast mechanism is based on two-photon excited fluorescence (TPEF) signal from NADH, FAD, keratin, melanin, and elastin fibers and on second harmonic generation (SHG) signal from collagen. These images were used as a basis for the mitochondrial clustering analysis.

Suction Blister Induction and cell isolation for single-cell RNA sequencing: All procedures were conducted according to an approved institutional review board protocol of the University of California, Irvine (HS No. 2018-4362), with written informed consent obtained from all patients. The donor skin sites were cleaned with ethanol wipes and 5 suction blisters (1 cm diameter) were created by applying a standard suction blister device. The blisters were unroofed and used half for melanocyte-keratinocyte transplant procedure. The rest of the blisters were incubated in trypsin for 15 minutes at 37° C., followed by mechanical separation and centrifugation at 1000 rpm for 10 minutes at 4° C. to pellet cells. Cells were washed with 0.04% UltraPure BSA: PBS buffer, gently re-suspended in the same buffer, and filtered through a 70 μm mesh strainer to create a single cell suspension. Cells were washed and viability was calculated using trypan blue. scRNA-seq was performed by the Genomics High Throughput Sequencing facility at the University of California, Irvine with the 10x Chromium Single Cell 3′ v2 kit (10× Genomics). None of the patients that were imaged overlapped with the cohort of patients that were analyzed by single cell RNA sequencing.

Patient Samples for RNAscope: All procedures were conducted according to an approved institutional review board protocol of the University of California, Irvine (HS No 2018-4362) with written informed consent obtained from all patients. Briefly, 2 mm biopsies were performed on lesional and nonlesional skin as part of punch grafting treatment for three patients. Control skin was acquired from tumor excision tips without notable pathology from patients without vitiligo. Skin samples were immediately frozen and embedded in OCT. Tissues were stored at −80° C. and cryosections (10 mm thick) of skin were collected on Fisherbrand Superfrost Plus microscope slides. Sections were dried for 60-120 minutes at −20° C. then used immediately or within 10 days. In situ hybridization was performed according to the RNAscope Multiplex Fluorescent Reagent Kit v2 (Cat. No. 320293). Briefly, slides were fixed in cold 4% PFA for 15 minutes then dehydrated in 50%, 70%, and 100% ethanol for 5 minutes each at room temperature (RT). H₂O₂ was applied for 10 minutes at RT and treated with protease IV for 30 minutes. C2 and C3 probes were diluted in C1 probes at a 1:50 ratio and incubated for 2 hours at 40° C. C1 probes were detected with TSA-fluorescein (Akoya Biosciences), C2 probes with Opal-620 and C3 probes with Opal-690 (Akoya Biosciences). Before mounting, DAPI was added to label the nuclei. Images were acquired using a Leica SP8 FALCON/DIVE (20× objective, 0.75 NA).

Statistical Analysis: Statistical comparisons of median B and B variability were conducted using linear mixed effects models in SAS JMP Pro 14. Variables such as patient number and imaging location were modeled as random effects. Whether an area of skin was lesional or non-lesional was modeled as a fixed effect when comparing metrics of mitochondrial clustering among patients. Whether an area of skin was sun-exposed or non-sun-exposed was modeled as a fixed effect when comparing metrics of mitochondrial clustering among healthy volunteers. The significance level for all statistics was set to α=0.05.

Mitochondrial Clustering: All image processing steps were performed in MATLAB using an approach established previously. Several masks were created and combined in order to isolate cytoplasmic autofluorescence. An SHG mask was primarily created to remove contributions from collagen and stromal autofluorescence at the interface of the epidermis and dermis. Contrast-limited adaptive histogram equalization (CLAHE) was applied to SHG images and features were subsequently segmented using Otsu's global thresholding. The SHG mask was finalized by applying a median filter to remove noise and taking the complement of the image to mask features corresponding to the segmented signal. Features corresponding to highly autofluorescent biomolecules such as keratin and melanin were masked using similar methods. CLAHE was applied to TPEF images and an Otsu's global threshold was calculated. Pixels with intensity values 1.5× greater than the Otsu's global threshold were segmented and masked. This empirically determined threshold was applied to all optical sections and was determined based on the propensity to remove highly autofluorescent signatures without masking pixels from intermediate cell layers which would not contain fluorophores such as keratin and melanin. The removal of nuclear and interstitial regions was achieved by applying 3 serial bandpass filters to contrast-limited adaptive histogram equalized TPEF images. Remaining features were segmented using Otsu's global thresholding. A circular mask with a 500-pixel diameter was created to remove dim image corner artifacts. Masks were finalized with the removal of objects less than 8-pixels in size. The final mask was applied to raw TPEF images, which were then subjected to a digital object cloning (DOC) process. The DOC process randomly fills any void pixels from the masking process with the signal that was identified as cytoplasm. No pixels are overwritten during this process and it is replicated 5 times. The average power spectral density (PSD) of the 5 DOC images was then computed and fitted with an equation of the form R(k)=Ak-β for spatial frequencies (k) less than 0.118 μm-1 (features smaller than 8.5 μm). The absolute value of the fitted exponent, β, represents the degree of mitochondrial clustering within the cytoplasm. Mitochondrial clustering was computed for optical sections ranging from the stratum corneum to the stratum basale. Depth-dependent metrics of mitochondrial clustering were computed for each stack of images. β variability represents the sample variance of β values as a function of depth and aims to capture depth-dependent changes in metabolism. Median β represents the median β value as a function of depth and aims to capture the overall level of metabolic activity.

Mitochondrial clustering was calculated in the same manner for single-cell analysis. Due to the relatively low levels of contrast in the basal layer of the epidermis, single cells had to be manually segmented. One optical section per patient region of interest was segmented for single cells. Approximately 5-10 single cells were masked per image. A total of 182 cells from lesional and 258 cells from non-lesional regions were included for analysis. All vitiligo patients included in the imaging studies were represented in the total cell populations. The heterogeneity level of the corresponding distributions was quantified using a previously established heterogeneity index, based on fitting a 2-Gaussian mixture model to each distribution. Briefly, the heterogeneity index, H, can be computed using the equation H=−Σ dp ln p, where i denotes each subpopulation, d denotes the absolute value of the difference between the median of a subpopulation and the median of the total population, and p denotes the Gaussian mixing proportion of the subpopulation. 2-Gaussian mixture models were derived using SAS JMP Pro 14 statistical software.

Processing and quality control of scRNA-seq data: Sequencing libraries were prepared using the Chromium Single Cell 3/v2 protocol (10× genomics). Sequencing was performed on Illumina HiSeq4000 platform (Illumina). FASTQ files were aligned utilizing 10× Genomics Cell Ranger 2.1.0. Each library was aligned to an indexed hg38 genome using Cell Ranger Count. Cell Ranger Aggr function was used to normalize the number of mapped reads per cell across the libraries. Patient B sample and lesional skin of Patient G sample did not have enough viable cells and was excluded from further analysis (FIG. 14). Quality control parameters were used to filter cells with 200-4000 genes with a mitochondrial percentage under 18% for subsequent analysis.

Integration and clustering analyses of scRNA-seq data: Integration and clustering of cells was performed using the scMC R package, which is an R toolkit for integrating and comparing multiple scRNA-seq experiments across different conditions. And scMC learns a corrected matrix, which is a shared reduced dimensional embedding of cells that preserves the biological variation while removing the technical variation. The data of each lesional and nonlesional skin of each patient were treated as one condition. Therefore, the input of the scMC is a list with 11 elements, with each element being one condition. The parameters used for this data are shown as follows: resolution=1; quantile.cutoff=0.5, similarity.cutoff=0.65. To identify cell clusters, principal component analysis (PCA) was first performed on the corrected matrix of scMC and the top 40 PCs with a resolution=1 were used to obtain 14 clusters for all the samples.

Calculation of signature score of a gene set: For gene scoring analysis, most gene sets were acquired from the MSigDB database (https://www.gsea-msigdb.org/gsea/msigdb/). Gene sets of metabolic pathways were from published literature. The AddModuleScore function in Seurat R package was then used to calculate the signature score of each gene set in each cell. The two-sided Wilcoxon rank sum test was used to evaluate whether there are significant differences in the computed signature scores between two groups of cells.

Cell-cell communication analyses: Recently, a new computational tool CellChat was developed to systematically infer and analyze intracellular communication from scRNA-seq data. CellChat infers the biologically significant cell-cell communication by assigning each interaction with a probability value (i.e., interaction score or weight) and performing a permutation test. CellChat models the probability of cell-cell communication by integrating gene expression with prior known knowledge of the interactions between signaling ligands, receptors and their cofactors including soluble agonists and antagonists, as well as co-stimulatory and co-inhibitory membrane-bound receptors. The intercellular communication networks for the nonlesional and lesional skin were separately inferred and then jointly analyzed using CellChat (version 1.1.0). The average expression of signaling genes per cell cluster was computed using the truncated mean, where 10% of expression levels were trimmed from each end of data. Since CellChat infers intracellular communications based on cell clusters, the interactions associated with cell clusters with very few cells were potentially artifacts. Thus, the inferred interactions associated with stressed keratinocyte population in nonlesional skin were filtered out because of the extremely low percent of stressed keratinocytes compared to other keratinocytes in nonlesional skin (FIG. 3D).

Pseudotime and trajectory analysis. The PHATE dimensional reduction of keratinocytes from all samples was performed by taking the shared low dimensional space obtained by scMC as an input. The parameters used in PHATE on the data are as follows: npca=30, t=3. When inferring pseudotemporal trajectory of keratinocytes, the PHATE space was used in the reduced dimensional space in Monocle 3. A principal graph is learnt by learn graph function with the parameters: minimal_branch_len=5, rann.k=18 and Euclidean_distance_ratio=2. Pseudotime values of cells were obtained once cells were ordered based on the learnt graph. In addition, the possible transitions between different cell subpopulations was also inferred using PAGA by using the PHATE space as a reduced dimensional space.

RNA velocity analysis: RNA velocity was calculated based on the spliced and unspliced counts as previously reported, and cells that were present in the pseudotemporal trajectory analysis were used for the analysis. The python implementation “scvelo” was used with PHATE space as an input.

Embodiments of the present invention can be freely combined with each other if they are not mutually exclusive.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1. A method of treating vitiligo in a patient in need thereof, said method comprising: topically administering a composition to a target area of skin tissue of the patient affected by vitiligo, wherein the composition is effective for inducing glycolysis and stimulating repigmentation in at least a portion of the target area of skin tissue administered the composition.

2.-4. (canceled)

5. The method of claim 1, wherein the composition comprises a biguanide.

6. The method of claim 5, wherein the biguanide is metformin.

7. The method of claim 5, wherein the biguanide is phenformin.

8. The method of claim 1, wherein the composition further comprises a pharmaceutically acceptable carrier.

9. The method of claim 1 further comprising co-administering a secondary therapeutic.

10. The method of claim 9, wherein the secondary therapeutic is a JAK inhibitor

11. The method of claim 9, wherein the secondary therapeutic is administered at the same time as the composition.

12. The method of claim 9, wherein the secondary therapeutic is administered at a time before the composition is administered.

13. The method of claim 9, wherein the secondary therapeutic is administered at a time after the composition is administered.

14.-26. (canceled)

27. A method of inducing repigmentation in skin lacking melanocytes of a patient, said method comprising: topically administering a composition to a target area of skin tissue of the patient, wherein the composition comprises a biguanide, the composition is effective for stimulating repigmentation in at least a portion of the target area of skin tissue administered the composition.

28.-29. (canceled)

30. The method of claim 27 wherein the biguanide is metformin or phenformin.

31. (canceled)

32. The method of claim 27, wherein the composition further comprises a pharmaceutically acceptable carrier.

33. The method of claim 27 further comprising co-administering a secondary therapeutic.

34. The method of claim 33, wherein the secondary therapeutic is a JAK inhibitor

35. The method of claim 33, wherein the secondary therapeutic is administered at the same time as the composition.

36. The method of claim 33, wherein the secondary therapeutic is administered at a time before the composition is administered.

37. The method of claim 33, wherein the secondary therapeutic is administered at a time after the composition is administered.

38.-44. (canceled)

45. A method of transplanting skin cells, said method comprising: harvesting melanocytes and keratinocytes from a portion of skin in a patient; subjecting the skin cells to a treatment, said treatment comprising a composition comprising a biguanide; and transplanting skin cells onto the patient.

46. The method of claim 45, wherein the biguanide is metformin or phenformin.

47. (canceled)
